(19) United States
(12) Patent Application Publication
(10) Pub. No.: US 2005/0085744 A1
(43) Pub. Date: Apr. 21, 2005

(54) Man-machine interfaces system and method, for instance applications in the area of rehabilitation

(75) Inventors: Fabrizio Beverina, Merate (IT); Giorgio Palmas, Milano (IT); Stefano Silvoni, Rovigo (IT)

Correspondence Address: SEED INTELLECTUAL PROPERTY LAW GROUP PLLC, 701 FIFTH AVENUE, SUITE 6300, SEATTLE, WA, 98104-7092, US

(73) Assignee: STMicroelectronics S.r.I., Agrate Brianza (IT)

(21) Appl. No.: 10/970,751

(22) Filed: Oct. 20, 2004

Related U.S. Application Data
(60) Provisional application No. 60/512,976, filed on Oct. 20, 2003.

(51) Int. Cl. A61B 5/00

(52) U.S. Cl. 600/558; 600/559; 600/544

(57) ABSTRACT

A system for developing a brain-computer interface (BCI), especially for use in rehabilitation, includes an audio-visual interface device for applying to a subject being examined stimuli eliciting event-related potentials and inducing brain reactions in said subject being examined. The system further includes an acquisition device for acquiring brain reaction signals (such as EEG traces) of the subject being examined synchronized with the stimuli and at least one processing device for processing the signals acquired via said acquisition device, The interface device, the acquisition device and the processing device comprise an integrated system. Preferably, the system uses a p300 signal as the event-related potential.

### BACKGROUND OF THE INVENTION

1. Field of the Invention

The present invention relates to techniques for man-machine interaction and has been developed with particular attention paid to its possible application in the area of rehabilitation techniques.

2. Description of the Related Art

Developing a system of man-machine interface, commonly referred to as brain-computer interface (BCI), entails developing an environment that will enable real-time interaction between the subject and the machine.

This means that such a system should possess at least the following characteristics:
an interface mechanism, comprising a communication protocol;
a data-acquisition system; and
a calculation system for pre-processing the signal and for its processing.

In real applications, it frequently happens that:
the characteristics are present in systems separate from one another that exchange information with long and cumbersome modalities; and
different application programs may be present in a single system that maintain the corresponding data in proprietary formats, which are difficult or even impossible to interpret.

The impossibility of using an integrated system that embraces the above characteristics frequently forces persons responsible for carrying out research to work in off-line mode. This means that data acquisition is temporally separate from the processing step so that it is not possible to provide the subject being tested with any feedback, this being an important element in the implementation of a BCI system.

In on-line mode, instead, the subject can receive a feedback from the system, and thanks to this peculiarity it is possible to model the interaction between the machine and the subject, contextualizing it within the framework known in the literature by the name of mutual learning (see, for example, J. del R. Millan, J. Mourino, F. Babloni, F. Cincotti, M. Varsta, J. Heilkonnen, “Local Neural Classifier For Eeg-Based Recognition Of Mental Tasks”, IEEE-INNS-ENNS International Joint Conference on Neural Networks, Jul. 24-27, 2000, Como, Italy), namely, the mechanism through which both the subject and the system learn specific skills for mutual communication.

In general, there exist numerous different approaches that can be used for implementing a BCI system. To limit our attention just to the ones which, in an essentially medical context, use electroencephalogram (EEG) signals, it is possible to name:
approaches that analyse slow cortical potentials (SCPs); see in this connection: J. Perelmouter, N. Birbaumer, “A Binary Spelling Device Interface With Random Errors”, IEEE Transactions on Rehabilitation Engineering, No. 2, vol. 8 (2000) 227-232, or else N. Birbaumer, N. Ghanayim, T. Hinterberger, I. Iversen, B. Kotchoubey, A. Kubler, J. Perelmouter, E. Taub, and H. Flor, “A Spelling Device For The Paralysed”, Nature, vol. 398 (1999), 297-298;
approaches that exploit de-synchronization of certain particular rhythms in EEG signals; see in this connection: D. J. McFarland, G. W. Neat, R. F. Read, J. R. Wolpaw, “An Eeg-Based Method For Graded Cursor Control”, Psychobiology, No. 1, vol. 21, (1993), 77-81, or else D. J. McFarland, L. M. McCane, S. V. David, J. R. Wolpaw, “Spatial Filter Selection For Eeg-Based Communication”, Electroenceph. Clin. Neurophy., vol. 103, (1997) 386-394;
approaches that exploit de-synchronization of the α and β rhythms in centro-parietal regions; see in this connection the article of Millán et al. already cited previously, and again: C. Guger, A. Schlöbgl, D. Walterspacher, G. Pfurtscheller, “Design Of An Eeg-Based Brain-Computer Interface (Bci) From Standard Components Running In Real-Time Under Windows”, Biomed. Technik, vol. 44, (1999) 12-16; and
approaches that envisage the use of the p300 signal; see in this connection: E. Donchin, K. M. Spencer, R. Wijesinghe, “The Mental Prosthesis: Assessing the Speed of p300-Based Brain-Computer Interface”, IEEE Transactions on Rehabilitation Engineering, Vol. 8, 2 (June, 2000) 174-179.

Albeit in the light of a known technique which is from many standpoints fertile and articulated, there exists the need to have available systems in which the interface, acquisition and processing devices will be completely integrated to provide a complete system for developing a BCI.

### BRIEF SUMMARY OF THE INVENTION

One embodiment of the present invention provides a system that will fully meet the need delineated previously.

In the currently preferred embodiment of the invention, interface, acquisition and processing devices are integrated for the purpose of providing a complete system for developing a BCI, with the possibility of exploiting accordingly both the peculiarities of the acquisition system and the experience acquired as regards the analysis, for example, of ERP-mediated traces (see in this connection S. Giove, F. Piccion, F. Giorgi, F. Beverina, S. Silvoni, “p300 off-line detection: a fuzzy-based support system”, WILF, Italian Workshop on Fuzzy Logic, Oct. 4-5, 2001, Milan, Italy).

Operation of the system is linked to the integrity of the cognitive functions of the subject being examined. Whilst the embodiment described in what follows by way of example pre-supposes the availability of some motor ability, albeit minimal, the solution according to the invention enables use thereof also on the part of subjects completely disabled from the motor and aphasic standpoint, i.e., totally incapable of communicating with the external environment.

### BRIEF DESCRIPTION OF THE SEVERAL VIEWS OF THE DRAWINGS

The invention will now be described, by way of non-limiting example, with reference to the annexed drawings, in which:

FIG. 1 represents an example of an ERP trace;

FIG. 2 is a schematic representation of an integrated system such as the one described herein;

FIG. 3 represents the acquisition of EEG data in the context of a system such as the one described herein;

FIG. 4 illustrates the connection logic of the modules making up the system described herein;

FIG. 5 represents an example of embodiment of a graphic interface in a system such as the one described herein;

FIG. 6, which is made up of two parts designated by a) and b), is a qualitative representation of some modalities of use of a system such as the one described herein;

FIG. 7 illustrates, at an elementary level, a neural-network architecture that can be used in a system such as the one described herein;

FIG. 8 represents the trend of a hyperbolic-tangent function; and

FIGS. 9 and 10 exemplify the results that may be achieved with a system such as the one described herein.

### DETAILED DESCRIPTION OF THE INVENTION

By way of foreword to the ensuing description, it will be necessary to recall that the tests for eliciting event-related potentials (ERPs), or endogens, contemplate a repeated stimulation of the subject being examined and the simultaneous recording of the EEG traces synchronized with the stimuli. These potentials may be recorded on the scalp only when the subject being examined selectively activates his own attention on a stimulus which he identifies as semantically relevant (target), or which he recognizes as deviant with respect to the other (non-target) stimuli. These potentials basically depend upon the context in which the target stimuli are supplied and are relatively independent of the physical characteristics of the stimulus.

The distribution on the cranial surface of an ERP component does not have a direct correspondence with the cerebral sites of its source. The ERPs supply precise information as regards the temporal succession of electro-physiological events correlated to different operations or phases of cognitive processes. Furthermore, they can be elicited with any type of sensorial modality.

The p300 signal is an event-related potential characterized by a wide symmetrical positive deflection (i.e., that does not present phenomena of lateralization on the scalp and is more evident in the derivations of the median line), said deflection being more represented in the centro-parietal regions of the scalp. It can be recorded only when the subject identifies a deviant stimulus, which is new or which takes on a particular semantic meaning.

The p300 signal is independent of the sensorial modality of the stimulus and can be evoked in different situations in which the subject has to perform mental operations.

The p300 signal is an electro-physiological index of perceptive processes and mnemic processes (i.e., relating to the memory), both short-term and long-term ones, which enable identification and classification of the stimulus. It is a manifestation of the cerebral activities that take place whenever the internal representation of the environment is to be updated.

The latency of this characteristic deflection is around 300 ms, but may range between 250 ms and 600 ms according to the type of stimulus and the difficulty of the task to be performed.

Said deflection is usually preceded by two stimulus-related deflections, “N1” and “P2”, and by an event-related component, “N2”. Normally, it is possible to note said deflections following upon the operation of temporal averaging on the set of EEG responses to the target stimuli received by the subject.

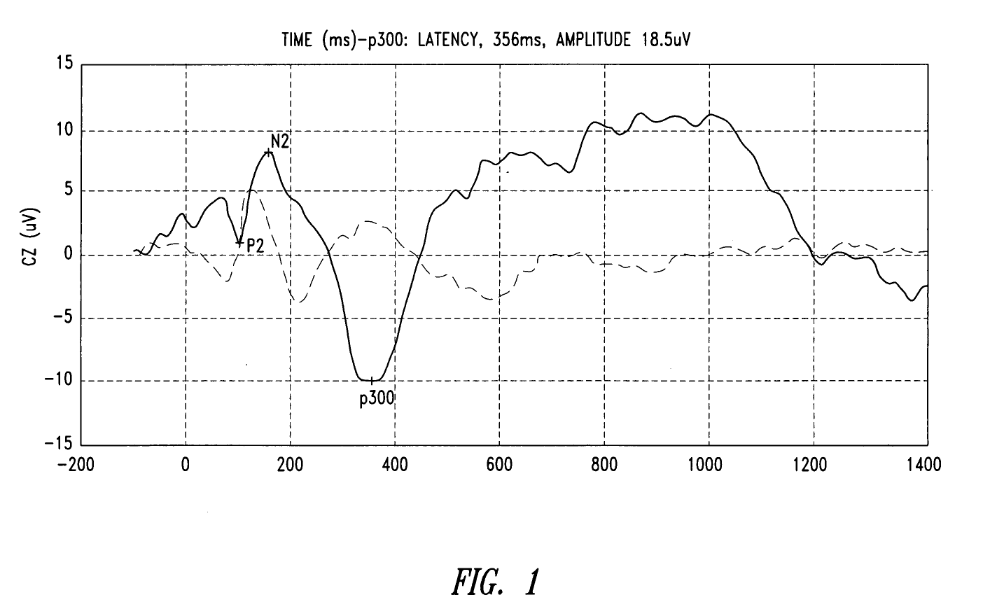

In particular, FIG. 1 provides the representation as a function of time (in milliseconds—latency: 356 ms; amplitude: 18.5 μV) of an ERP trace. This has been obtained via averaging of numerous responses to target stimuli; highlighted are the principal components (P2, N2, p300 or p3) and the latency of the p300 deflection with the corresponding amplitude; the dashed trace corresponds to the averaging of the responses to non-target stimuli.

Even though the averaging operation will enable a significant observability of the p300 potential, its identification in EEG traces for individual responses proves far more complicated, on account of the electro-encephalographic activity superimposed on the ERP components.

The system described in what follows carries out recognition of this potential, by analysing directly the individual epochs recorded in concomitance with the stimuli.

The system in question is made up of different hardware and software modules integrated with one another, amongst which a stimulator apparatus S for elicitation during the ERP test, and an amplifier A of EEG signals that is specific for low frequencies.

The main components of the system are:
the acoustic and visual stimulator S, equipped with ear-phones C and electrodes T affixed to a head H of a subject, with associated thereto a control keypad K;
a computer PC1, which controls acoustic stimulation of the subject via the ear-phones and visual stimulation via a monitor;
an amplifier SA for amplifying EEG signals;
a computer PC2, which handles the acquisition, processing, and display of the EEG signals;
a software module (usually resident on the computer PC1), which enables preparation of the stimuli and their presentation to the subject; and
an applicational software module (usually resident on the computer PC2), which controls acquisition of the EEG signals.

All the components/modules referred to above are commercial products that are available from NeuroSoft Inc.

The system is moreover supported by a Matlab environment ML and corresponding scripts for data processing (MathWorks), as well as by an application package AA, which manages the connection between acquisition and processing of the data and the graphic interface for feedback to the subject (this, for example, may be the product NSAcqLink program supplied by STMicroelectronics).

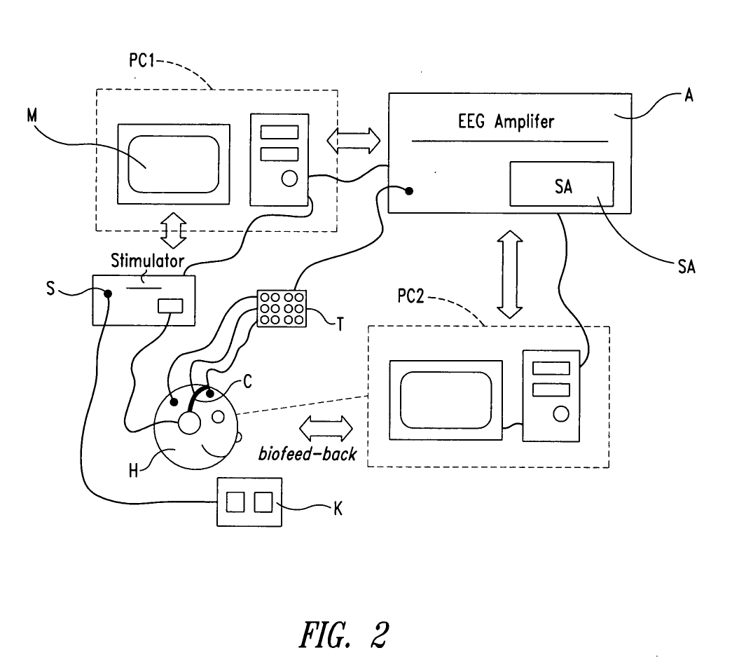

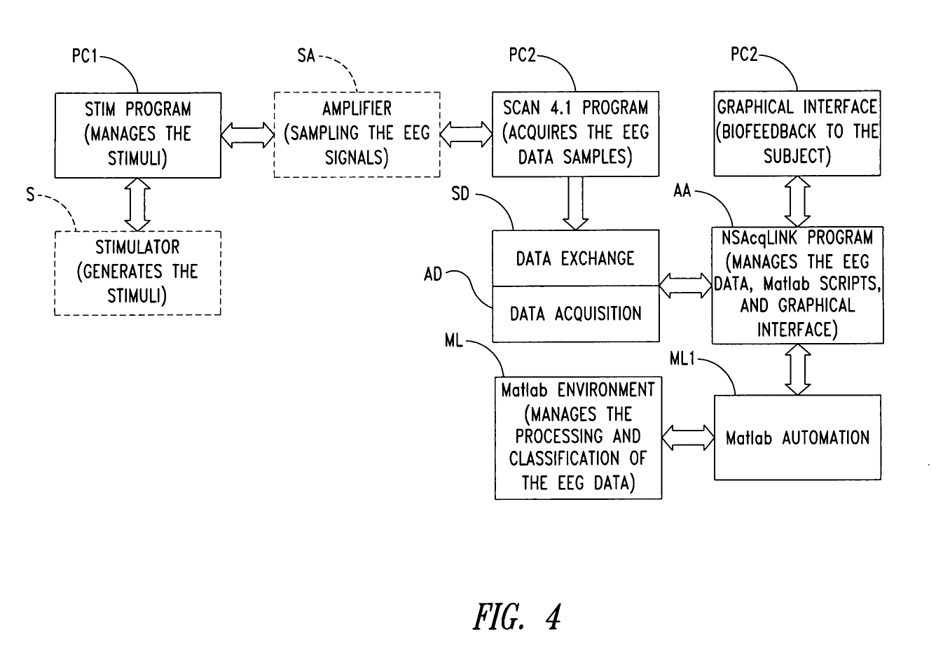

The physical connection of the hardware components is represented in FIG. 2, whilst the logic connection of the software components is represented in FIG. 4, where the reference ML1 designates the function of automation of the Matlab environment, and the references SD and AD designate the functions of data exchange and data acquisition, respectively.

During a generic test, the subject is administered a random sequence of predefined acoustic or visual stimuli, with fixed inter-stimulus intervals. Said stimuli are controlled by the program for managing the stimulation resident on the computer PC1, which, at each stimulus, generates a trigger signal that enables the amplifier SA to detect the occurrence of the event. Simultaneously, the computer PC2 samples and records the EEG signals coming from the electrodes mounted on the scalp of the subject being examined (Fz, Cz, Pz).

Usually an additional electrode (EOG, Electro-oculogramme) is used for verifying the ocular movements, in particular blinking. The trigger signal thus enables synchronized recording of the EEG signals with the onset of the stimuli.

Usually, the electrodes used in this type of test are located in the median line of the scalp, a region in which it is possible to record the cognitive potentials of highest intensity. In particular, Fz relates to the frontal area, Cz to the central one, and Pz to the parietal one.

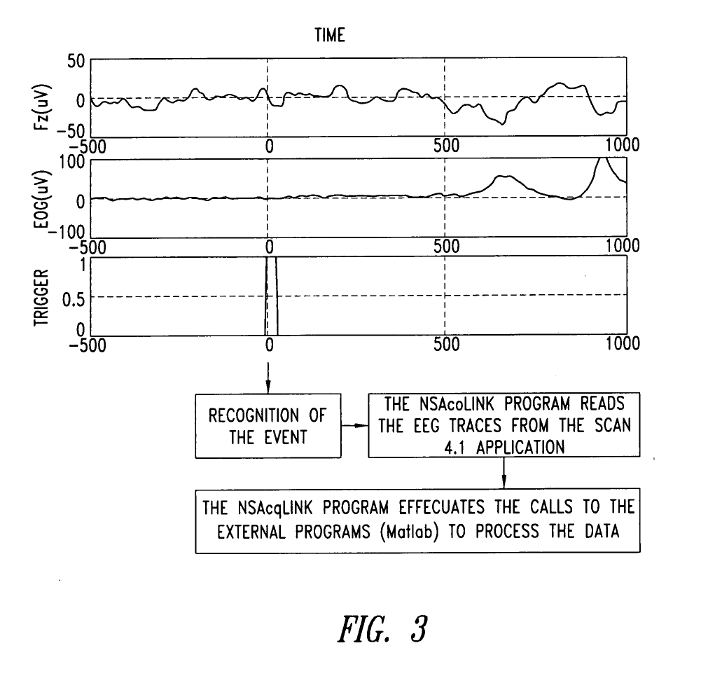

FIG. 3 offers a representation of the acquisition of the EEG data synchronized with the trigger signal. In particular, Fz is the signal for the frontal channel, EOG is the signal for the electro-oculogramme, whilst trigger is the signal that enables synchronization of the traces with the stimuli.

Typically, the data are gathered in epochs of 1500 ms, after which they are transferred to the processing and classification algorithms.

By means of a software exchange mechanism, i.e., a specific Dynamic Link Library (DLL), the data are gathered into epochs of 1.5 s, 0.5 s before the stimulus and 1 s after the stimulus, and then transferred to the module AA. Subsequently, the single epochs (single-sweep) of the data acquired are passed to the Matlab environment ML for their processing and classification.

The output of the classification algorithm is then read by the main program M, which handles the graphic interface for the bio-feedback to the subject.

The modularity that characterizes the organization described herein bestows on the system certain particular features:
modularity and flexibility: some components can be replaced with similar components, without altering the system; this applies in particular for the purposes of classification of the p300 signal;
the possibility of pre-defining the stimuli to be administered to the subject and of changing the graphic interface for the feedback enable a diversification of the tests on the BCI for scientific purposes;
the possibility of saving the EEG data on backup files enables working in off-line mode with a different computer, without the need to use all the equipment present in the laboratory.

The system or working environment described enables use of a test protocol consisting of a paradigm for eliciting the p300 signal and two stages referred to as learning stage and testing stage. The protocol in question enables the following objectives to be achieved:
setting up a system of man-machine interaction using the mutual-learning approach, in which, through the management of specific internal parameters, the classification system is adapted to the peculiarities of the EEG traces in response to the stimuli typical of the subject being examined, this also causing the subject, according to his particular characteristics, to adapt to the classification system, by making an effort to concentrate attention on the task to be performed;
helping the subject, through a (visual) bio-feedback, to concentrate on the task that he has been assigned; and
verifying the performance of one or more algorithms for classification of the p300 signal.

More specifically, in the learning stage the subject is administered, for example, a test that is in part similar to the so-called classic Odd-Ball paradigm. The test proves to be more complex than the ones known in the literature in order to satisfy the typical constraints of the BCI context. This enables the classification system to determine the main characteristics of the p300 signal of the subject, and these are then used in the subsequent recognition stage (see, in this connection, the article by S. Giove et al., referred to previously).

The type of stimulation administered to the subject may consist of four key words (vocal stimuli received by the subject through the ear-phones), if the acoustic mode is used, or else by four arrows indicating four possible directions, if the visual mode is used; in either case, the stimulations are presented with a random sequence and with an inter-stimulus interval of 2.5 s. In the case of visual signals:
A=“Forwards” or arrow up “ [symbol] ” (25%);
D=“Right” or arrow to the right “ [symbol] ” (25%);
I=“Backwards” or arrow down “ [symbol] ” (25%);
S=“Left” or arrow to the left “ [symbol] ” (25%);
Consequently, the sequences assume a random form, such as, for example: . . . A, D, A, I, S . . . D, D, I, D, A, I . . . with the percentages of occurrence specified.

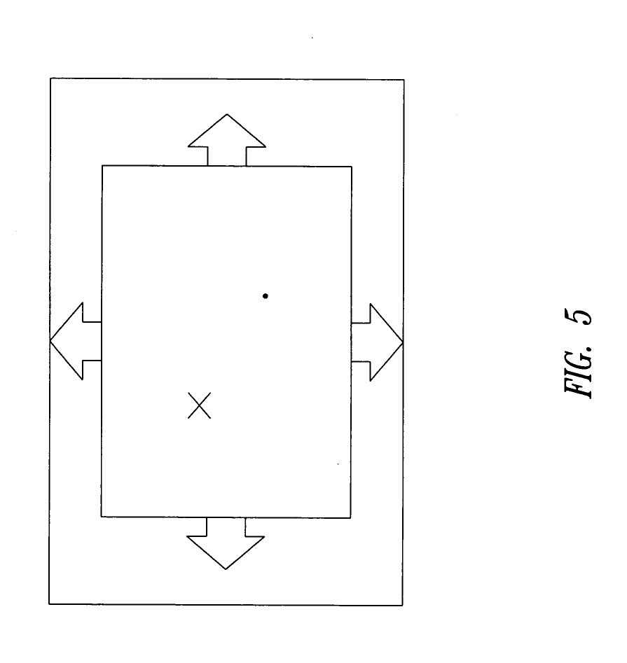

The task, for the subject being examined, is the displacement of an object (a point) displayed on the monitor of the ScanPC for achieving a target (the cross, see FIG. 5). For this purpose, the subject must concentrate his attention on the stimuli that enable displacement of the object in the direction of the target; these stimuli will be defined as “significant” or “target” stimuli, whilst the remaining stimuli will be defined as “non-significant” or “non-target” stimuli. Specifically, FIG. 5 represents the display of the graphic interface: initial position of the object (point) and of the target (cross) on the screen of the computer PC2.

The significant displacements may depend upon a predefined path, or else can be decided upon during the test by the subject himself, but in any case are signalled to the system by depression of a key. In either case, during the learning stage it is always possible to determine which stimulus of the four possible ones is significant.

At the end of the test, there is available a set of single-sweep traces, i.e., individual epochs of EEG traces synchronized with the stimuli and divided into two classes:
traces representing EEG activity linked to the presumed elicitation of a p300 signal, characteristic of the subject being examined; and
traces representing EEG activity where an elicitation of the p300 signal is presumed not to be present.

At the end of this step, the system seeks to learn the specificity of the p300 signal, characteristic of the subject being examined; for this purpose, a first training stage of the chosen recognition algorithm is started, through analysis of the two classes of traces.

In the testing stage proper, the subject H and the system interact in a non-constrained manner; i.e., the subject selects, from the four stimuli proposed, the one that is most significant for him to achieve the target, without communicating it to the system, whilst the system evaluates, in the EEG activity associated to each stimulus, the presence or the absence of the p300 signal.

The subject is then asked to concentrate his attention on performing the same task illustrated previously: displacement of the point towards the target.

For each stimulus administered, the system classifies the single-sweep traces, highlighting the presence or absence of the p300 signal: it may be noted that the classification occurs in real time, stimulus by stimulus, without any activity of averaging on the traces.

The displacement of the object, in contrast with what occurs in the learning stage, is determined by the classification system, i.e., on the basis of the evaluation of the EEG response to the stimulus made by the classifier.

The presence of favourable situations or of situations of conflict between the direction chosen by the subject and the result of the classification, with consequent correct or erroneous movement of the object, generates a visual bio-feedback on the subject.

In summary:
at each stimulus received, the recognition algorithm evaluates the presence or otherwise of the p300 signal in the corresponding single-sweep traces;
the system is able to know a priori the type of stimulus just administered to the subject;
if a p300 has been identified, then the system moves the object, on the screen, in the direction corresponding to the stimulus (known beforehand: forwards, backwards, right or left);
if a p300 has not been identified, then the object remains stationary.

Hence, if the subject elicits a recognizable p300, he sees as a result the displacement of the point in one of the four directions. If said signal is recognized in a point corresponding to the stimulus on which the subject was concentrating his attention, then the displacement will come about in the direction of the target (reinforcement, positive bio-feedback); otherwise, the displacement will be in a direction opposite to that of the target (denial, negative bio-feedback).

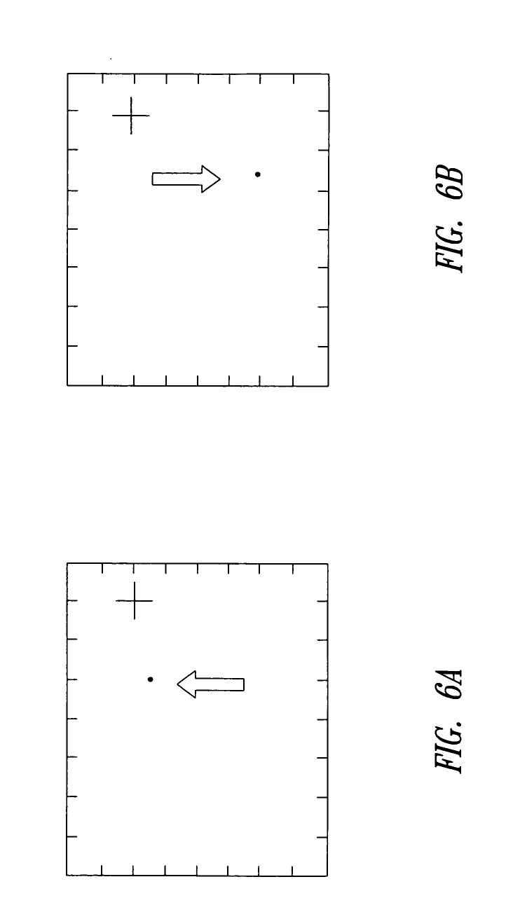

FIG. 6 is a qualitative representation of the two types of bio-feedback in the case of recognition of a p300 signal: a) positive, the object approaches the target; b) negative, the object moves away from the target.

The estimation of the performance of the system considers the following quantities:
NP₃₀₀=number of significant (or target) stimuli received by the subject;
Nₙₒₙ₋ₚ₃₀₀=number of non-significant (or standard) stimuli received by the subject;
N_{TP}=number of correct recognitions of the responses to target stimuli;
N_{TN}=number of correct recognitions of the responses to standard stimuli;

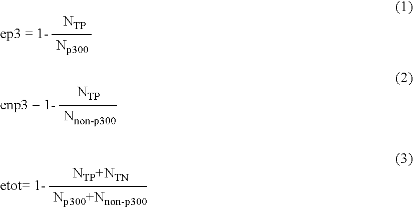

A further estimation of the quality of the test can be made by analysing the errors corresponding to the non-significant stimuli (2), defined as false positives. Via this evaluation it is possible to understand whether the number of displacements of the object, due to correct classifications (FIG. 6-a), enables the subject to perform his own task successfully and without particular difficulties.

The following inequality takes into account the relationship between the correct responses and the wrong ones in such a way that, in the limit case (equality), the number of movements towards the target will counterbalance the centrifugal movement due to erroneous classification of the non-relevant stimuli:
probApproach≧probRecession 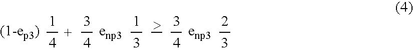
whence:
1−eₚ₃≧eₙₚ₃ (5)

From the off-line analysis of the single-sweep traces gathered during the testing stage, it is possible moreover to gain further information for a new training of the recognition algorithm of the p300 signal.

In effect, in the hypothesis of mutual learning, carrying out multiple learning and testing trials should generate performances that improve as the number of trials increases. A graph that gives the total error (3) according to the number of tests or trials carried out, can illustrate said improvement.

Classification of the on-line (single-sweep) traces raises various problems of a critical nature:
the characteristics of the p300 signal depend to a large extent upon the subject and upon the elicitation paradigm;
the cognitive potential is found to be superimposed on the background EEG activity; frequently, the signal-to-noise ratio is rather low;
the presence of ocular artefacts (in particular, blinking) renders interpretation of the EEG traces in response to the stimuli difficult;
the p300 signal can be evoked also by unexpected stimuli, in the experiment in question, one of the three “non-significant” arrows for achieving the objective.

On the one hand, then, it is advantageous to identify a methodology of analysis that will enable attenuation of the contribution to the cognitive potential due to the artefacts and other EEG activities. On the other hand, it is useful to identify a testing protocol for elicitation of the p300 which will limit, as far as possible, the intra-individual variabilities. As regards, instead, inter-individual differences, adaptation of the system is made to the requirements of the individual person; i.e., the information that is most relevant for an effective classification is extracted from the traces of a subject and subsequently used in the testing stage on the same subject.

The processing and classification techniques so far used for analysis of the traces envisage the following fundamental steps:
filtering via ICA (Independent Component Analysis), with the aim of increasing the signal-to-noise ratio in the single-sweep traces;
extraction of the characteristics typical of the signal (features); and
classification via a neural network and re-training for following the evolution of the subject (mutual learning).

The ICA technique proposes finding the independent signals sⱼ (sources), from the linear composition of which the measured variables xᵢ are generated:

$${x}_{1} = {\sum }_{j = 1}^{N} {a}_{ij} {s}_{j} . \qquad ( 6 )$$

which, in vector notation, can be written as follows:
x=As (7)

It is a matter, then, of finding the unmixing matrix B (B≅A⁻¹), by solving the system ŝ=Bx (in which both s and B are unknowns) such that the sⱼ are as independent as possible (according to the cost function chosen ad hoc).

By statistical independence is meant:
f(y₁, . . . yₘ)=f₁(y₁)·f₂(y₂) . . . ·fₘ(yₘ) (8)
where the yᵢ are stochastic variables, f(y₁, . . . , yₘ) is the joint-probability distribution, and the fᵢ(yᵢ) are the marginal probability distributions.

To do this, the following assumptions are made (see, in this connection, A. Hyvarinen, Erkii Oja, “Independent Component Analysis, a Tutorial”, IEEE Neural Networks, 1999, and A. Hyvarinen, “Survey on Independent Component Analysis”, http://www.cis.hut.fi/˜aapo):
the mixture of the signals is instantaneous and non-convolutive; i.e., the coefficients aᵢⱼ are real numbers and not transfer functions (in z⁻¹) of the sources;
the number N of the components of the signal sj is smaller than or equal to the number of the signals detected* xᵢ; in the case in point, we have chosen the number of the sources s equal to the number of the electrodes, i.e., 4;
the components sⱼ have a non-gaussian distribution (at least all except one); this restriction is obligatory in so far as a linear composition of random gaussian variables (zero-average) is still a gaussian (for gaussian variables uncorrelation and independence are equivalent, given that the variables are completely defined by their first-order and second-order statistics), a fact that renders the estimations of the number and of the characteristics of the gaussian components impossible.

Finally, to solve the system, the condition is imposed that the variables/signals s are statistically independent.

To do this, we have available various possibilities given by the various measurements of statistical independence that are found in the literature.

Amongst these, it is possible to mention, in the first place, the measurement based upon the minimization of the mutual information I between the stochastic variables sᵢ, with i=1 . . . . N, as follows:

$$I ( {s}_{1} , {s}_{2} , {s}_{3} , … , {s}_{N} ) = {{\sum }_{1}}^{N} H ( {s}_{i} ) - H ( s ) \qquad ( 9 )$$

where H is the entropy for a discrete stochastic variable of possible values aᵢ, defined as follows:

$$H ( Y ) = - {\sum }_{i} p ( Y = {a}_{i} ) log p ( Y = {a}_{i} ) \qquad ( 10 )$$

The mutual information yields a measurement of the dependence between the stochastic variables, taking into account the entire structure of the variables, and not only the covariance. It is in fact well known that if the variables sⱼ are statistically independent, their mutual information is zero, and vice versa, if the mutual information is zero, they are statistically independent If the mutual information is interpreted using code theory, the terms H(sᵢ) yield the length of the code for sᵢ, and H(s) yields the length of the code when s is considered as a single variable. It follows that, by minimizing the mutual information, those variables are sought which together do not provide information; i.e., if all the variables are encoded separately, the length of the code created by encoding all the variables together does not increase; therefore, the variables prove to be independent, as confirmed, on the other hand, by the articles authored by Hyvarinen et al., already referred to previously.

Alternatively, it is possible to resort to the method which is based upon the consideration that, if two signals are statistically independent, then the covariances, cov{sᵢ(t)sⱼ(t+τ)} must all be zero, ∀i≠j, ∀τ; the unmixing matrix B is hence calculated by imposing that the variables s(t)=Bx(t) will have a diagonal autocovariance for every value of time delay.

Thus the problem of simultaneous diagonalization of M covariance matrices (where M are the time instants that are taken into consideration) is solved by using the method proposed by Yeredor (see for example A. Yeredor, “Approximate Joint Diagonalization Using Non-Orthogonal Matrices”, Proceedings of ICA2000, p.p. 33-38, Helsinki, June, 2000, or again A. Yeredor, “Non-Orthogonal Joint Diagonalization in the Least-Squares Sense with Application in Blind Source Separation”, IEEE Trans. On Signal Processing, vol. 50, No. 7, pp. 1545-1553, July, 2002).

Recourse to the first method currently appears preferential, in so far as it yields better results notwithstanding the following limitations:
the hypothesis that the number of sources into which the signal is broken down is smaller than or equal to the number of the acquired signals imposes that, by taking three EEG and EOG derivations, it is possible to break down the cerebral signal just into three components of a cerebral origin and one component due to the EOG; this may prove not altogether satisfactory in certain applications;
the decomposition does not present a fixed physical/spatial order; this implies that it is not possible to know, a priori, which of the sources will correspond to the p300 signal; the choice of the derivation can be made manually by the operator, although it appears preferable to be able to choose automatically the source presenting the smallest deflection around 300400 ms;
not necessarily is the unmixing matrix, calculated with the training signals, the best also for the subsequent signals; in stationary conditions (which are practically guaranteed by a good experimental set-up and by a “good” subject) the decomposition matrix proves to be always the same; in actual fact, instead, frequently the subject is distracted and the decomposition matrix chosen does not prove to be the best one.

Passing now to the description of the features extracted for trace classification, once the unmixing matrix B has been determined from a set of traces, the source that contains the p300 signal is chosen. Next, using the same unmixing matrix, the selected source is extracted from the single-sweep traces and reduced to a set of features, which enable a synthetic description of the information content of the signal.

The above features seek to highlight certain peculiarities of the cognitive potentials contained in the traces.

In a particularly advantageous embodiment, 78 features have been chosen in all, amongst which:
minimum, and index of the minimum;
maximum, and index of the maximum between 0-700 ms after the stimulus;
power normalized in four time intervals;
sum normalized in the same four time intervals;
sub-band analysis via wavelet decomposition on 5 octaves with bi-spline orthogonal wavelet (this family has been chosen because it has a shape similar to the evoked response—see, in this connection: R. Quian Quiroga, “Obtaining Single Stimulus Evoked Potentials With Wavelet Denoising”, Von Neumann Institute for computing, Julich Germany, 2001); a very interesting characteristic of the wavelet transform is the possibility of carrying out a time/frequency analysis of the signal; a characterization of the signal in time with respect to the power intensities corresponding to the delta, theta, alpha, beta and gamma frequencies thus proves to be simple and computationally far from burdensome—see in this connection: Strang Nguyen, “Wavelets and Filter Banks”, Wellesley, Cambridge Press, 1996;
zero crossing; and
total time in which the curve has dropped below zero.

The features corresponding to a single-sweep trace, in all 78, constitute the input for the classifier described in what follows.

The global performance of the interface is linked, in the ultimate analysis, to the degree of mutual learning between the system and the user. In this sense, the design choices for the classifier take into consideration the high degree of variability in performance that can be put down to the particular psychophysical state of the user.

Once acceptable error values have been reached (in terms of false positives and false negatives), the possible improvement in performance is entrusted to the stage of mutual learning.

In a preferred way, the classification is implemented through a neural network; the architecture adopted and the learning algorithm are optimized during off-line sessions.

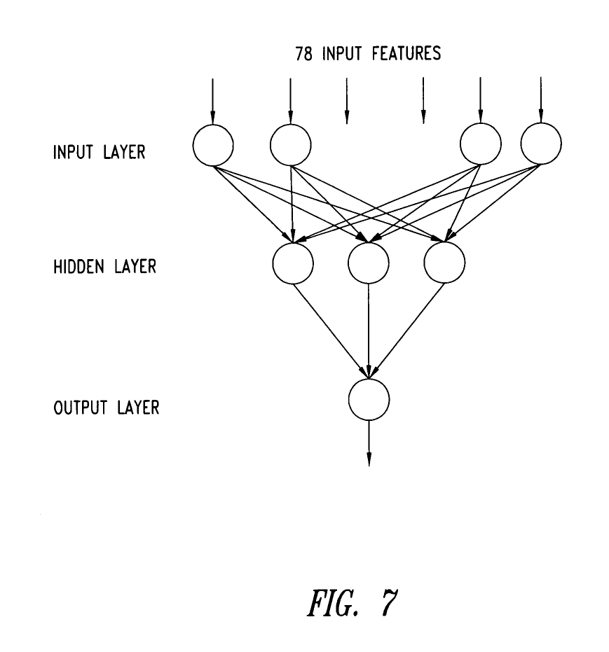

As illustrated in FIG. 7, the architecture adopted is made up of three layers. The choice of said preferred structure has been dictated by the other than high number of examples on which the network operates (on average 500) and by the need to minimize the degrees of freedom represented by the number of weights to be trained. Said network is designated, for reasons of convenience, by 78_3_1. In some tests, there has been likewise used a network with a four-layer architecture, which can be identified as 78_4_2_1.

The main parameters are:

| Net type | 78_3_1 | 78_4_2_1 |
|---|---|---|
| Weights | 237 | 322 |
| Units | 82 | 85 |

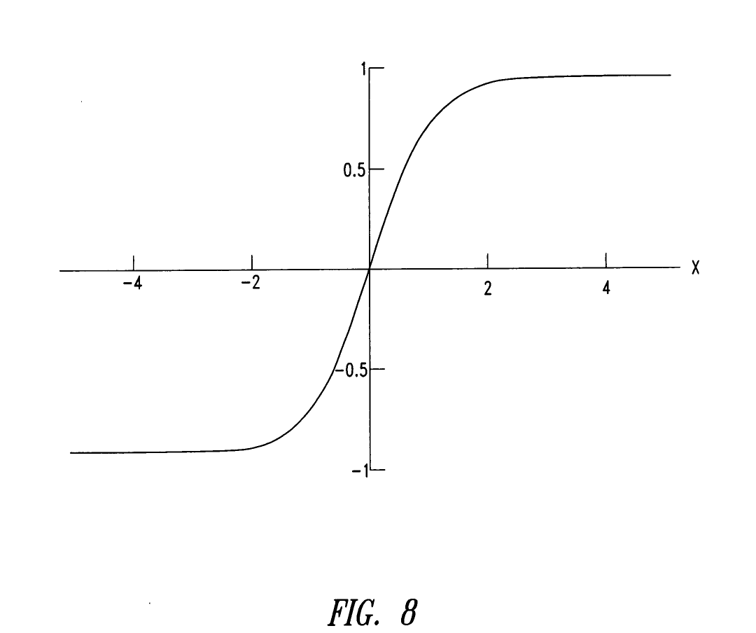

The function of activation used for all the units is the hyperbolic tangent, the output of which is comprised in the interval [−1, 1] (see FIG. 8):

$$Tanh ( x ) = \frac{{e}^{x} - {e}^{- x}}{{e}^{x} + {e}^{- x}} \qquad ( 11 )$$

The initial values of the weights and of the thresholds have been chosen in the interval [−0.5, 0.5]; this interval has been kept somewhat reduced in order to prevent phenomena of saturation of the weights.

The algorithm used is the well-known back-propagation algorithm in the variant which envisages addition, in the step of back-propagation of the errors, of a quantity, moment, which renders the network more sensitive to the mean variations of the error surface.

In brief,

$$Δ {w}_{ij} ( t + i ) = η \frac{\partial E}{\partial {w}_{ij}} + α Δ {w}_{ij} ( t ) \qquad ( 12 )$$

where η indicates the learning rate, E the cost function, and α the moment.

As regards training of the network, the set of the examples has been split into three separate sub-sets:
Training Set: used for training the network;
Validation Set: used, during the training stage, for verifying the goodness achieved in terms of generalization; and
Testing Set: used only in the testing stage for validation of the network.

Normally, given the set of examples, the validation set and the testing set each represent approximately 10% of the total. With the analysis of the traces, the definition of a set of parameters is achieved, amongst which η, α, and the number of epochs, which have contributed to developing the procedure for on-line classification of the traces.

After a series of tests were conducted, both to check correct operation of the integrated system and to verify the usability of the testing protocol, the system described herein was tested on a subject affected by multiple sclerosis (a 37-year-old male).

Initially, the system was trained for recognition of the p300 signal on the basis of 8 recordings corresponding to the learning stage. Subsequently, after each test, the network weights were updated by means of the learning algorithm. In all the sessions, a paradigm for elicitation via visual stimuli, i.e., via stimuli of the same type as the biofeedback to the subject, was used.

The results of this individual test, consisting of 19 testing steps with the network 78_3_1 and 5 testing steps with the network 78_4_2_1, are encouraging, even though testing was carried out at a purely experimental level.

In particular, out of 5 sessions in which the network 78_3_1 was used, the subject succeeded in completing the task assigned (reaching the target with the object), as likewise in 4 sessions using the network 78_4_2_1.

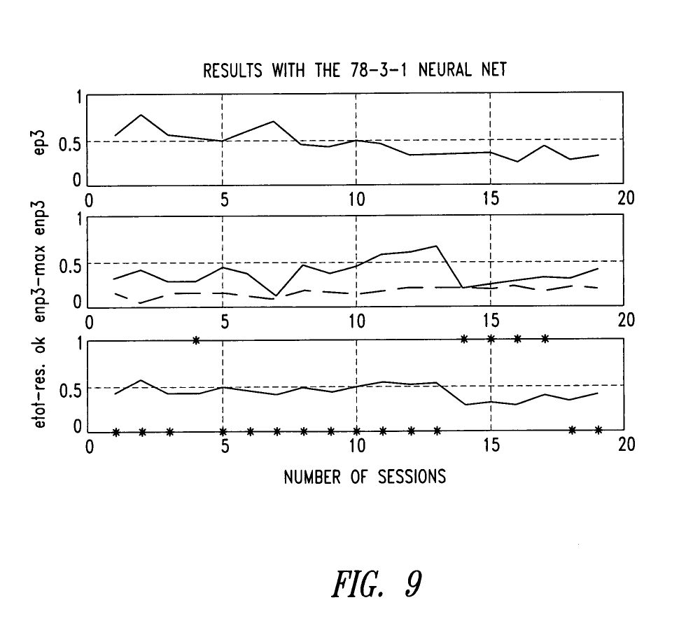

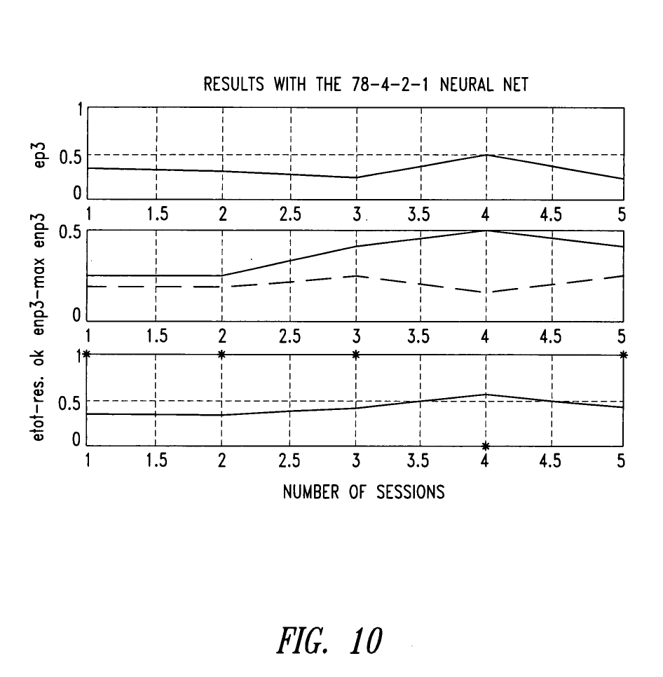

FIGS. 9 and 10 illustrate the trend of the errors (1), (2), (3) and the empirical measurement of the upper limit for false positives (4), according to the number of sessions conducted.

In particular, FIG. 9 represents the trend of the errors corresponding to the testing sessions, during which the network 78_3_1 was used; the sessions completed successfully are highlighted with the symbol “*” in a position corresponding to the value 1.

FIG. 10 represents, instead, the trend of the errors corresponding to the testing sessions, during which the network 78_4_2_1 was used; also here the sessions completed successfully are highlighted with the symbol “*” in a position corresponding to the value 1.

The working environment used here is based upon two fundamental facts: the stimulation of the subject and the recognition, on the part of the system, of the significant stimuli. In fact, the subject is asked to concentrate on certain particular stimuli, which at the moment of testing have a given meaning. On the other hand, the system makes the attempt, via the EEG signals, to discriminate the responses of the subject to significant (target) events from all the non-significant (standard) events.

As may be appreciated, this idea can be generalized for the purposes of man-machine communication. In particular, the type and the mode of stimulation can be adapted according to the applicational requirements, whereas the basic principle in all cases remains correct recognition of the presence or absence of the p300 signal.

This generalization opens the way to multiple applications in the medical and social fields for persons with serious difficulties of communication, such as, for example, tetraplegic subjects (in particular, the most serious ones, in which the possibilities of communication are reduced to the minimum).

The most important critical factors encountered in this type of test relates to the choice of the system for processing and classification of the EEG traces, as well as its adaptation to the subject being examined; in this case, an artificial neural network has been used.

Thanks to the flexibility of the integrated system, it is possible to develop the BCI by substituting and experimenting various criteria of classification.

It will be appreciated that one of the main aspects that can be considered stable is the possibility of working in on-line mode with a modular and flexible system both in terms technical requirements and in terms of scientific experimentation. Such a system constitutes the starting base necessary for the development and implementation of a BCI that may be useful as communication device for persons suffering from serious physical handicaps.

The solution described herein consequently enables planning and development of an integrated system for man-machine interaction, for the purposes of a potential use as communication device for subjects with serious physical handicaps. In particular, it is possible to integrate the hardware and software resources appropriately both for elicitation of a cognitive potential of interest and for definition of a procedure of calculation for on-line recognition of said potential.

It will be appreciated that one of the most significant peculiarities of the system described herein consists in the implementation of an instrument that is able to operate on line, with the corresponding applicational advantages, this fact setting it off significantly from the majority of other technologies in use, which, instead, employ methods of off-line analysis.

The system may be used for carrying out real-time tests on the recognition of an ERP, i.e., of the so-called “p300” signal that can be observed in EEG traces. The system uses both an experimental protocol purposely designed and tested on a set of subjects and a procedure for recognition of the signal based upon soft-computing techniques, such as adaptive neural networks, capable of optimizing their own parameters on the basis of the different responses of the subjects.

It is therefore evident that, without prejudice to the principle of the invention, the details of implementation and the embodiments may vary even significantly with respect to what is described and illustrated herein, without thereby departing from the scope of the present invention as defined by the claims that follow.

All of the above U.S. patents, U.S. patent application publications, U.S. patent applications, foreign patents, foreign patent applications and non-patent publications referred to in this specification and/or listed in the Application Data Sheetare incorporated herein by reference, in their entireties.

1. A system for developing a brain-computer interface (BCI), comprising:
an interface device for applying to a subject being examined stimuli eliciting event-related potentials and inducing brain reactions in said subject being examined;
an acquisition device for acquiring brain reaction signals of said subject being examined synchronized with said stimuli; and
a processing device for processing said signals acquired via said acquisition device, wherein said interface device, said acquisition device and said processing device comprise an integrated system.

2. The system of claim 1 wherein said acquisition device is configured for acquiring EEG traces as said brain reaction signals synchronized with said stimuli.

3. The system of claim 1, further comprising a recording device for recording said signals acquired via said acquisition device.

4. The system of claim 1, wherein said acquisition device is positioned on a scalp of said subject being examined.

5. The system of claim 1, further comprising a stimulus generator for selectively generating stimuli selected out of a group consisting of semantically relevant target stimuli for said subject being examined and stimuli deviant with respect to non-target stimuli for said subject being examined.

6. A system for developing a brain-computer interface (BCI), comprising an interface device for applying to a subject being examined stimuli eliciting at least one event-related potential and inducing brain reactions in said subject being examined, said interface device configured for eliciting a p300 signal as said at least one event-related potential.

7. The system of claim 6, further including:
an acquisition device for acquiring brain reaction signals of said subject being examined synchronized with said stimuli; and
a processing device for processing said signals acquired via said acquisition device, wherein said interface device, said acquisition device and said processing device comprise an integrated system.

8. The system of claim 6, further comprising an acquisition device for detecting said p300 signal as preceded by stimulus-related deflections, and an event-related component.

9. The system of claim 8 wherein said acquisition device is configured for acquiring EEG traces and detecting said deflections within said EEG traces.

10. An integrated system for developing a brain-computer interface (BCI) the system comprising:
an acoustic stimulator and a visual stimulator for applying to a subject being examined stimuli eliciting event-related potentials and inducing brain reactions in said subject being examined;
a control unit for controlling acoustic stimulation and visual stimulation as applied to said subject being examined via said acoustic stimulator and said visual stimulator;
an acquisition device for acquiring brain reaction signals of said subject being examined synchronized with said stimuli; and
a computer for managing acquisition of said brain reaction signals via said acquisition device and processing said signals acquired.

11. The system of claim 10, further including a display unit for displaying said signals acquired.

12. The system of claim 10 wherein said control unit includes a computer program product loaded therein which enables preparation of said stimuli and their presentation to the said subject being examined.

13. The system of claim 10 wherein said computer includes a computer program product loaded therein which controls acquisition of said brain reaction signals.

14. A method of developing a brain-computer interface (BCI), comprising the steps of:
applying to a subject being examined stimuli eliciting event-related potentials and inducing brain reactions in said subject being examined;
acquiring brain reaction signals of said subject being examined synchronized with said stimuli;
processing said signals acquired via said acquisition device; and
conducting at least one test sequence by administering to said subject a random sequence of predefined stimuli, with predetermined inter-stimulus intervals.

15. The method of claim 14 wherein said predefined stimuli include acoustic stimuli.

16. The method of claim 14 wherein said predefined stimuli include visual stimuli.

17. The method of claim 14, further including the step of generating, in correspondence with said stimuli, trigger signals enabling detection of an occurrence of a corresponding event.

18. The method of claim 14, further including the step of detecting EEG signals coming from a scalp of said subject being examined.

19. The method of claim 18, further including the step of detecting EEG signals from a median line of the scalp of said subject being examined.

20. The method of claim 18, further including the step of detecting EEG signals from at least one of a frontal area, a central area, and a parietal area of the scalp of said subject being examined.

21. The method of claim 14, further including the step of verifying ocular movements of said subject being examined.

22. The method of claim 21, further including the step of verifying eye blinking of said subject being examined.

23. The method of claim 14, further including the step of acquiring said brain reaction signals in epochs.

24. The method of claim 23 wherein said epochs have a length of about 1500 ms.

25. The method of claim 14, further including the step of acquiring said brain reaction signals in epochs extending both before and after a respective stimulus.

26. A method of developing a brain-computer interface (BCI), comprising the steps of:
applying to a subject being examined acoustic stimuli eliciting event-related potentials and inducing brain reactions in said subject being examined; and
acquiring brain reaction signals of said subject being examined synchronized with said stimuli, wherein said acoustic stimuli include a set of key words presented to said subject being examined with a random sequence.

27. The method of claim 26, wherein said random sequence of acoustic stimuli has an inter-stimulus interval of about 2.5 s.

28. A method of developing a brain-computer interface (BCI), comprising the steps of:
applying to a subject being examined visual stimuli eliciting event-related potentials and inducing brain reactions in said subject being examined; and
acquiring brain reaction signals of said subject being examined synchronized with said stimuli, wherein said visual stimuli include a set of arrows displayed to said subject being examined with a random sequence.

29. The method of claim 28 wherein said random sequence of visual stimuli has an inter-stimulus interval of about 2.5 s.

30. A method of developing a brain-computer interface (BCI), comprising applying to a subject being examined stimuli eliciting at least one event-related potential and inducing brain reactions in said subject being examined, wherein said at least one event-related potential is a p300 signal.

31. A method of developing a brain-computer interface (BCI), comprising the steps of:
applying to a subject being examined stimuli eliciting event-related potentials and inducing brain reactions in said subject being examined; and
acquiring brain reaction signals of said subject being examined synchronized with said stimuli, the brain reactions signals including:
traces representing EEG activity linked to a presumed elicitation of a p300 signal in said subject being examined; and
traces representing EEG activity where an elicitation of the p300 signal is presumably absent in said subject being examined.

32. The method of claim 31, further including the steps of:
displaying to said subject being examined an object adapted to move consistently with the applied stimuli;
at each stimulus applied to said subject being examined, detecting whether said p300 signal is present in the corresponding single-sweep traces; and
if a p300 signal is detected, moving said object displayed in a direction corresponding to the stimulus applied; and
if a p300 signal is not detected, leaving said object stationary.
